(19) United States
(12) Patent Application Publication
(10) Pub. No.: US 2018/0345115 A1
(43) Pub. Date: Dec. 6, 2018

(54) Techniques for Displaying User's Physical State

(71) Applicant: Under Armour, Inc., Baltimore, MD (US)

(72) Inventors: Nathan Dau, Baltimore, MD (US); Jeffrey Allen, Baltimore, MD (US); Mark A. Oleson, Baltimore, MD (US)

(21) Appl. No.: 15/607,814

(22) Filed: May 30, 2017

(51) Int. Cl. A63B 71/06; A41D 1/00; A41B 11/00; A41D 1/08; A41D 1/06; A63B 24/00; A61B 5/00

(52) U.S. Cl. CPC A63B 71/0622; A41D 1/00; A41B 11/00; A41D 1/08; A41D 1/06; A63B 24/0062; A63B 2230/00; A61B 5/6807; A61B 5/443; A63B 2071/0661; A63B 2024/0065; A63B 2225/50; A61B 5/6804

(57) ABSTRACT

A a patch is provided for application against the skin of a user. The patch includes a substrate, a material detector, a memory, a comparator, a three state indicator and a power source. The material detector is disposed on the substrate, has a contact portion disposed so as to contact the skin, and can generate a material signal based on an amount of material contacting the contact portion. The memory has a priori material data stored therein. The comparator outputs a compared signal based on a comparison of the material signal and the a priori material data. The three state indicator provides an indication of the state of the user based on the compared signal, wherein the indication being selected from one of the group consisting of a first indication of a first state, a second indication of a second state and a third indication of a third state. The power source provides power to the three state indicator.

### BACKGROUND

The present invention generally relates to indicate system for evaluating a user's physical state.

There exists a need for a device and method for detecting and indicating a user's physical state based on material loss.

### BRIEF SUMMARY OF THE DRAWINGS

The accompanying drawings, which are incorporated in and form a part of the specification, illustrate example embodiments and, together with the description, serve to explain the principles of the invention. In the drawings:

FIG. 1 illustrates a user running with an activity tracker;

FIG. 2 illustrates a user using a patch in accordance with aspects of the present invention;

FIG. 3 illustrates a bottom side view of a patch in accordance with aspects of the present invention; and

FIG. 4 illustrates a top side view of a patch in accordance with aspects of the present invention.

### DETAILED DESCRIPTION

### OVERVIEW

An aspect of the present invention is drawn to a patch for use with application against the skin of a user. The patch includes a substrate, a material detector, a memory, a comparator, a three state indicator, and a power source. The material detector disposed on the substrate and having a contact portion disposed so as to contact the skin. The material detector operable to generate a material signal based on the amount of material contacting the contact portion. The memory having a priori material data stored within. The comparator operable to output a compared signal based on the comparison of the material signal and the a priori material data. A three state indicator operable to provide an indication of the state of the user based on the compared signal, the indication being selected from one of the group consisting of a first indication of a first state, a second indication of a second state and a third indication of a third state. The power source operable to provide power to the three state indicator.

### EXAMPLE EMBODIMENTS

Fitness planners are a popular method of creating training regimens with a large variety of goals such as losing weight, building muscle, increasing strength, or endurance training. In general, a training regimen that is created by a fitness planner will instruct a user to perform an activity for a given amount of time based on their current physical state and goals.

For example, suppose a person who is in average physical shape would like to use a fitness planner to create a training regimen so that they may run a five kilometer race three months away. The training regimen would then instruct the user to perform an activity such as strength training, a three, five or seven kilometer run, or sprints for each training day leading up to the five kilometer race. Since the daily activity in the training regimen is set in advance, there is no way to take into account the physical state of the user while training, which may lead to the user overexertion and physical harm.

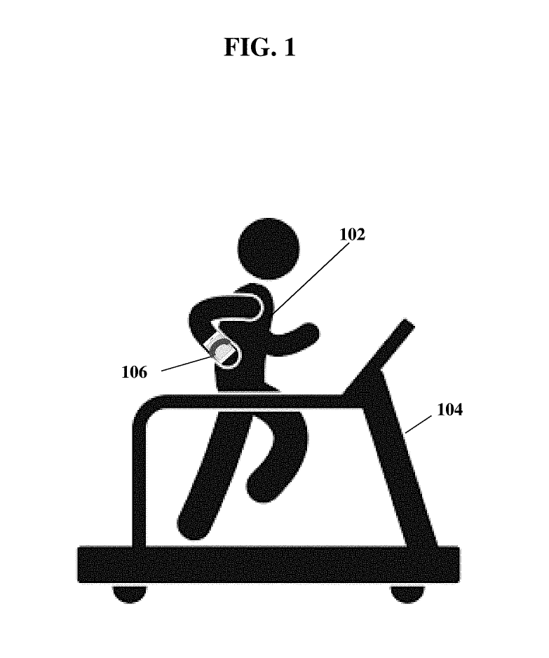

FIG. 1 illustrates a user running as dictated by a training regimen created using a conventional fitness planner.

The figure includes a user 102, a treadmill 104, and a phone 106.

User 102 runs on treadmill 104. User 102 uses phone 106 to track parameters associated with running on treadmill 104. Non-limiting examples of tracked parameters include step count, time of exercise, calories burned, etc.

In operation, user 102 is following a training regimen that was created in advance using a conventional fitness planner that does not consider the physical state of the user. In this example embodiment, the training regimen that was created instructs user 102 to run for five miles. At this time, user 102 begins running on treadmill 104 and uses phone 106 to track the distance they have run.

In this example, since the training regimen was created in advance, suppose user 102 is instructed to run five miles independent of their physical state. Further, suppose that user 102 has insufficient electrolytes and sodium (Na) levels for optimal performance in the five mile run. As user 102 runs, he will lose even more sodium and their physical state will decline rapidly. For purposes of discussion, let the physical state of user 102 deteriorate so much that user 102 is only be able to run two miles instead of five miles.

In this case, the training regimen will not compensate for user 102 failing to run the entire five miles, which will lead user 102 to fall behind their regimen. Further, each time user 102 fails to complete the predetermined amount of activity; their deficiency is compounded until they are unable to keep up with their training regimen at all.

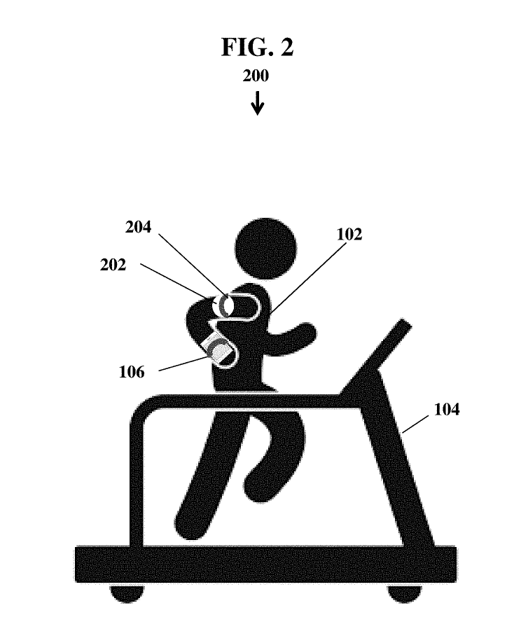

A patch in accordance with aspects of the present invention will quickly and easily inform a user of their state of readiness so as to help them prepare for an activity. Aspects of the present invention will now be described with reference to FIGS. 2-4.

FIG. 2 illustrates a user using a patch 202 in accordance with aspects of the present invention.

The figure includes user 102, treadmill 104, phone 106, a patch 202, and an article of clothing 204.

Patch 202 indicates the physical state of user 102.

Article of clothing 204 applies pressure in order to keep patch 202 pressed against the skin of user 102. Article of clothing 204 may be selected from one of the group consisting of an arm sleeve, a leg sleeve, a shirt, a pair of shorts, or a pair of pants and socks. In this example embodiment, article of clothing 204 is a sleeve, and in the rest of this example embodiment will be referred to as sleeve 204.

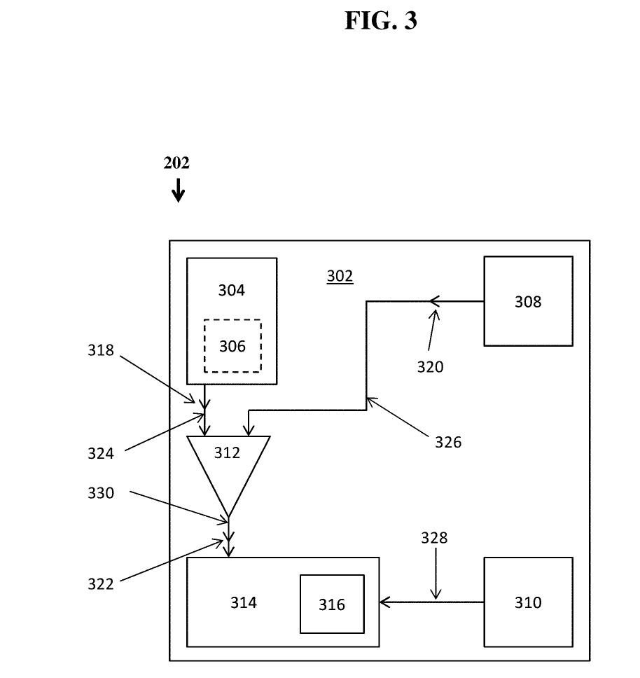

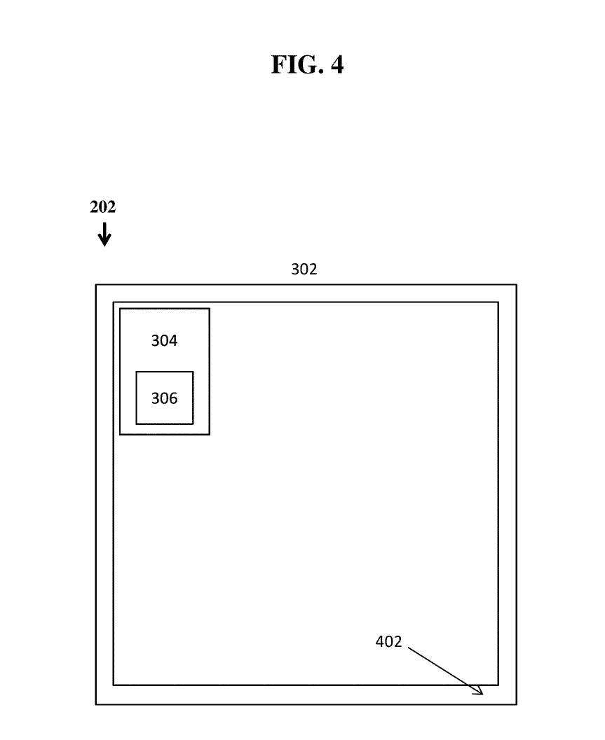

The operation of patch 202 will now be described with additional reference to FIGS. 3-4.

FIG. 3 illustrates a top side view of patch 202 of FIG. 2.

As illustrated in the figure, patch 202 includes a substrate 302, a material detector 304, a memory 308, a battery 310, a comparator 312, and a multi-state indicator 314. Material detector 304 further includes a contact portion 306. Multi-state indicator 314 further includes a light emitting diode (LED) 316.

Substrate 302 is the base of patch 202 on which material detector 304, memory 308, battery 310, comparator 312, and multi-state indicator 314 are mounted.

Material detector 304 generates material signal 318 based on an amount of material that is in contact with contact portion 306. Material detector 304 additionally transmits material signal 318 to comparator 312, via communication line 324. The material that is detected by material detector 304 may be one of the group consisting of sodium (NA), potassium (K), water (H₂O), ammonia (NH₃), potential hydrogen (pH), or lactic acid (LAC). In this example embodiment, the material detected by material detector 304 is sodium.

Contact portion 306 contacts the skin of user 102 of FIG. 2. In FIG. 3, contact portion 306 is illustrated as a box created by a dashed line to illustrate its placement underneath material detector 304. The location and operation of contact portion 306 will be further discussed in FIG. 4.

Memory 308 stores a priori material data that includes associations of material amounts with respect to mass loss levels. For purposes of discussion, mass loss may be considered a difference in body mass between two times. The main contributors to mass loss include loss of water, waste materials (e.g., sweat) and fat during aerobic and anaerobic respiration.

A priori data may be data previously generated by the user, or it may be data pre-loaded on memory 308 by the manufacturer, or it may be data previously generated by other users. The a priori data may include a lookup table or a database that references one or more associations between a detected material and a mass loss level. A priori data may also include functional relationships between two or more data.

Using raw a priori data or functional relationships between data may provide an output that corresponds to one or more aspects of the user's performance including, but not limited to, readiness, fitness, fatigue, and recovery time. For purposes of discussion, an example embodiment will be described wherein the aspect of the user's performance will be readiness.

In some embodiments, memory 308 additionally transmits stored a priori material data as material signal 320 to comparator 312, via communication line 326.

The a priori material data stored by memory 308 may be material data obtained from previous measurements taken from a user. Since the amount of a material detected will vary from person to person, the nominal amount of material in the system of each different user will be different as well. As such, initial measurements of an amount of material in the system of a user must be taken in order to generate a baseline value for which future measurements may be compared.

When a user checks their physical state, the amount of material detected will be stored for future reference. Each time a user checks their physical state, additional material data will be obtained, which can be averaged with previous measurements to create a more accurate baseline.

Additionally, the a priori data stored by memory 308 may be predetermined values that are associated with loss rates of a given material. In this case, the amount of material detected in the system of a user will be the baseline material amount for future measurements.

In this example embodiment, the a priori data that is stored by memory 308 is associated with mass loss levels of sodium detected by material detector 304, in other embodiments, the a priori data may be associated with mass loss levels of any one of the group consisting of sodium, potassium, water, ammonia, pH, or lactic acid.

Battery 310 supplies power to multi-state indicator 314, via line 328.

Comparator 312 compares material signal 318, from material detector 304, and material signal 320, from memory 308, in order to generate a compared signal. Comparator additionally transmits the compared signal as mass loss level signal 322 to multi-state indicator 314, via communication line 330.

In order to generate mass loss level signal 322, comparator 312 will compare the amount of material detected in the system of user 102 to the initial amount of material detected to determine how much material has been lost while performing an activity. The amount of material lost can then be compared to the predetermined mass loss rates stored as a priori information by memory 308 in order to determine the physical state of user 102.

In this example embodiment, suppose that: if user 102 is determined to have lost 20% of an initial amount of sodium that was detected, then user 102 will be considered to be in a high readiness state; whereas if user 102 is determined to have lost between 21% and 80% of the initial amount of sodium that was detected, then user 102 will be considered to be in a moderate readiness state; and whereas if user 102 is determined to have lost more than 81% of the initial amount of sodium that was detected, then user will be considered to be are in a low readiness state.

Multi-state indicator 314 provides an indication of one of a plurality of possible states of user 102 of FIG. 2 based on mass loss level signal 322. The indication provided to user 102 may be selected from one of the group consisting of a an image, a sound, a color, a vibration and combinations thereof. In a working example embodiment, multi-state indicator 314 is a three state LED, able to illuminate as red, yellow or green.

Multi-state indicator 314 may indicate the first state by turning LED 316 red, the second state by turning LED 316 yellow, and the third state by turning LED 316 green. In this example embodiment, the first state indicates that user 102 is in a high readiness state, the second state indicates that user 102 is in a moderate readiness state, and the third state indicates that user 102 is in a low readiness state.

In this example, material detector 304, contact portion 306, memory 308, comparator 312, and multi-state indicator 314 are illustrated as individual devices. However, in some embodiments, at least two of material detector 304, contact portion 306, memory 308, comparator 312, and multi-state indicator 314 may be combined as a unitary device.

Further, in some embodiments, at least one of memory 308 and comparator 312 may be implemented as a computer having tangible computer-readable media for carrying or having computer-executable instructions or data structures stored thereon. Such tangible computer-readable media can be any available media that can be accessed by a general purpose or special purpose computer. Non-limiting examples of tangible computer-readable media include physical storage and/or memory media such as RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. For information transferred or provided over a network or another communications connection (either hardwired, wireless, or a combination of hardwired or wireless) to a computer, the computer may properly view the connection as a computer-readable medium. Thus, any such connection may be properly termed a computer-readable medium. Combinations of the above should also be included within the scope of computer-readable media

LED 316 may be illuminated as red, yellow, or green by multi-state indicator 314.

FIG. 4 illustrates a bottom side view of patch 202 of FIG. 2.

As illustrated in the figure, patch 202 includes substrate 302, material detector 304, contact portion 306, and an adhesive layer 402.

Contact portion 306 contacts the skin of user 102 of FIG. 2.

Adhesive layer 402 adheres substrate 302 to the skin of user 102 of FIG. 2. A non-limiting example of adhesive layer 402 includes a layer of a pressure sensitive adhesive.

Referring back to FIG. 2, in operation, user 102 is following a training regimen that was created using a fitness planner. Suppose that in this example embodiment, the training regimen that was created instructs user 102 to run for five miles. Before beginning to run, user 102 decides that they would like to monitor their physical state while running In order to monitor their physical state, user 102 places patch 202 on their arm, and then they firmly press down on the top side of patch 202. In this example embodiment, user 102 placed patch 202 on their arm, in other embodiments, user 102 may place patch 202 on their leg, shoulder, or chest.

Briefly referring to FIG. 4, when user 102 places patch 202, contact portion 306 is contacting their skin. When user 102 applies pressure to the top side of patch 202, it activates adhesive layer 402, due to its adhesive being pressure sensitive. Once the adhesive activates, adhesive layer 402 is able to keep contact portion 306 against the skin of user 102.

Referring back to FIG. 2, since user 102 is going to be running five miles, there is a chance that patch 202 will not adhere to their skin once they begin to sweat. Patch 202 falling off of user 102 while running is not only problematic, but also interferes with user 102 to be able to monitor their physical state. To prevent patch 202 from falling off, user 102 places sleeve 204 on their arm, on top of patch 202. In this manner, sleeve 204 is able to apply pressure to patch 202 for the duration of the five mile run, further preventing patch 202 from falling off and allows user 102 to continuously monitor their physical state while running.

Referring to FIG. 3, once user 102 has placed patch 202 on their arm, applied pressure, and placed sleeve 204 on their arm to ensure that contact portion 306 is touching their skin, material detector 304 is able to detect the amount of sodium in the system of user 102, via contact portion 306.

Once material detector 304 detects the amount of sodium in the system of user 102, it generates material signal 318 based on the detected amount, which it then transmits to comparator 312, via communication line 324. The amount of sodium in the system of user 102 before starting to run will then be used by comparator 312 as a baseline for future measurements made while user 102 is runs.

At this time, user 102 begins their five mile run, and patch 202 begins to monitor their physical state. As described above, material detector 304 detects the amount of sodium in the system of user 102, via contact portion 306. Material detector 304 generates material signal 318, based on the detected amount of sodium, which is then transmitted to comparator 312. Memory 308 accesses the stored a priori data in order to locate data that is associated with loss levels of sodium. Once the data is located, memory 308 transmits the loss level data as material signal 320 to comparator 312, via communication line 328.

At this time, comparator 312 has received material signal 318, which contains the detected levels of sodium in the system of user 102, and material signal 320, which contains the a priori data associated with mass loss levels of sodium. First, comparator 312 will compare the detected amount of sodium to the baseline amount in order to determine how much sodium user 102 has lost. Since user 102 just began running, comparator 312 finds that user 102 has only lost 5% of the initial amount of sodium in their system.

Next, comparator 312 compares the amount of sodium lost to the loss level data in order to determine the state of user 102. Since user 102 has lost less than 20% of the initial amount of sodium in their system, comparator 312 determines that user 102 is in a high readiness state. Comparator 312 then generates mass loss level signal 322 based on the physical state of user 102, which it then transmits to multi-state indicator 314, via communication line 330.

Once multi-state indicator 314 receives mass loss level signal 322, it instructs LED 316 to turn green. Referring back to FIG. 2, once user 102 sees that LED 316 is green, they know that they are in a high readiness state and will continue their five mile run. Patch 202 continues to monitor the physical state of user 102 until a later time.

Referring to FIG. 3, after user 102 has run two miles, patch 202 continues to generate and transmit material signal 318 to comparator 312, based on the detected levels of sodium in the system of user 102. Simultaneously, memory 308 also generates and transmits material signal 320 to comparator 312. Comparator 312 compares the detected amount of sodium to the baseline amount and finds that user 102 has lost 25% of the initial amount of sodium in their system.

Next, comparator 312 compares the amount of sodium lost to the loss level data in order to determine the state of user 102. Since user 102 has lost between 21% and 80% of the initial amount of sodium in their system, comparator 312 determines that user 102 is in a moderate readiness state. Comparator 312 then generates mass loss level signal 322 based on the physical state of user 102, which it then transmits to multi-state indicator 314. After receiving mass loss level signal 322, multi-state indicator 314 turns LED 316 yellow in order to indicate to user 102 that they are in a moderate readiness state.

Referring back to FIG. 2, once LED 316 turns yellow, user 102 knows that they have lost a significant amount of sodium while running. Since LED 316 is yellow, user 102 knows that they are not yet in a low readiness state, and as such, they continue running while patch 202 continues to monitor their physical state.

Once user 102 has run a total of three miles, patch 202 continues to monitor the amount of sodium in the system of user 102. Referring to FIG. 3, as described above, material detector 304 generates and transmits material signal 318 to comparator 312, while memory 308 also generates and transmits material signal 320 to comparator 312.

Suppose that in this example embodiment, when comparator 312 compares the amount of sodium in the system of user 102 to the baseline amount of sodium, it finds that user 102 has lost 70% of the initial amount of sodium that was detected. At this time, when comparator 312 compares the amount of sodium lost to the loss level data, it finds that user 102 is still in a second state, but are nearing the third physical state marked by the loss of more than 81% of the initial amount of sodium detected.

As such, comparator 312 generates mass loss level signal 322, which it then transmits to multi-state indicator 314. Once multi-state indicator 314 receives mass loss level signal 322, it turns LED 316 red in order to indicate to user 102 that they have transitioned from a moderate readiness state to a low readiness state.

Referring to FIG. 2, once LED 316 turns orange, user 102 knows that they have lost a significant amount of sodium while running and that their physical state is diminishing and will soon be in a poor physical state. Since user 102 is nearing the end of their five mile run and are not yet in a poor physical state, they decide to continue running in order to try and complete their five mile run.

Referring to FIG. 3, once user 102 has run a total of four miles, material detector 304 generates material signal 318 based on the detected levels of sodium in the system of user 102, which it then transmits to comparator 312 and memory 308 generates material signal 320 based on stored a priori data, which it then transmits to comparator 312.

At this time, when comparator 312 compares the amount of sodium in the system of user 102 to the baseline amount of sodium, it finds that user 102 has lost 85% of the initial amount of sodium that was detected. Once comparator 312 compares the amount of sodium lost to the loss level data from memory 318, it finds that user 102 is in a third physical state. Comparator 312 then generates and transmits mass loss level signal 322 to multi-state indicator 314. Once multi-state indicator 314 receives mass loss level signal 322, it turns LED 316 red in order to indicate to user 102 that they have entered a third state.

Referring to FIG. 2, once LED 316 turns red, user 102 knows that they have lost a significant amount of sodium and are in a poor physical state. At this time, user 102 decides to stop running instead of trying to finish their five mile run.

A problem with conventional systems and methods for using a fitness planner or training regimen is that there is now way to account for the physical state of a user. If a user is in a poor physical state before beginning a difficult activity, it is possible that they may hurt themselves in attempting to finish the activity.

Aspects of the present invention include a patch for application against the skin of a user in order to monitor their physical state. The patch is able to monitor the amount of material in the system of a user as they perform a physical activity. As the user performs an activity, the patch is able to detect the amount of material that the user has lost.

The amount of material lost by a user can then be compared to a priori mass loss level data to determine the physical state of the user. By quickly and easily monitoring their physical state by way of a multi-state LED, a user can perform an activity safely by not over exerting themselves or continuing on with an activity if they are in a poor physical state.

The foregoing description of various preferred embodiments have been presented for purposes of illustration and description. It is not intended to be exhaustive or to limit the invention to the precise forms disclosed, and obviously many modifications and variations are possible in light of the above teaching. The example embodiments, as described above, were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed as new and desired to be protected by Letters Patent of the United States is:

1. A patch for application against the skin of a user, said patch comprising:
a substrate;
a material detector disposed on said substrate and having a contact portion disposed so as to contact the skin, said material detector being operable to generate a material signal based on an amount of material contacting said contact portion;
a memory having a priori material data stored therein;
a comparator operable to output a compared signal based on a comparison of the material signal and the a priori material data;
a three state indicator operable to provide an indication of the state of the user based on the compared signal, the indication being selected from one of the group consisting of a first indication of a first state, a second indication of a second state and a third indication of a third state; and
a power source operable to provide power to said three state indicator.

2. The patch of claim 1, further comprising a pressure sensitive adhesive layer disposed on said substrate so as to adhere said substrate against said skin.

3. The patch of claim 2, wherein said material detector is operable to generate the material signal based on a concentration of the material contacting said contact portion.

4. The patch of claim 3, wherein said material detector is operable to generate the material signal based on a concentration of the material selected from one of the group consisting of K, Na, H₂O and combinations thereof.

5. The patch of claim 4,
wherein said memory has a priori material data that includes associations of material concentrations with respective mass loss levels, and
wherein said comparator is operable to output the compared signal as a mass loss level signal.

6. The patch of claim 5, wherein said three state indicator comprises an LED operable to provide the indication selected from one of the group consisting of a red indication of the first state, a yellow indication of the second state and a green indication of the third state.

7. The patch of claim 1, wherein said material detector is operable to generate the material signal based on a concentration of the material contacting said contact portion.

8. The patch of claim 7, wherein said material detector is operable to generate the material signal based on a concentration of the material selected from one of the group consisting of K, Na, H₂O and combinations thereof.

9. The patch of claim 8,
wherein said memory has a priori material data that includes associations of material concentrations with respective mass loss levels, and
wherein said comparator is operable to output the compared signal as a mass loss level signal.

10. The patch of claim 9, wherein said three state indicator comprises an LED operable to provide the indication selected from one of the group consisting of a red indication of the first state, a yellow indication of the second state and a green indication of the third state.

11. The patch of claim 10, further comprising an article of clothing supporting said substrate such that when worn by the user, said article of clothing maintains said contact portion in contact with the skin.

12. The patch of claim 11, said article of clothing comprises one of the group consisting of an arm sleeve, a leg sleeve, a shirt, a pair of shorts, a pair of pants and socks.

13. The patch of claim 1, wherein said material detector is operable to generate the material signal based on a concentration of the material selected from one of the group consisting of K, Na, H₂O and combinations thereof.

14. The patch of claim 1,
wherein said memory has a priori material data that includes associations of material concentrations with respective mass loss levels, and
wherein said comparator is operable to output the compared signal as a mass loss level signal.

15. The patch of claim 1, wherein said three state indicator comprises an LED operable to provide the indication selected from one of the group consisting of a red indication of the first state, a yellow indication of the second state and a green indication of the third state.

16. The patch of claim 1, further comprising an article of clothing supporting said substrate such that when worn by the user, said article of clothing maintains said contact portion in contact with the skin.

17. The patch of claim 16, said article of clothing comprises one of the group consisting of an arm sleeve, a leg sleeve, a shirt, a pair of shorts, a pair of pants and socks.
